(19) United States
(12) Patent Application Publication
(10) Pub. No.: US 2024/0417358 A1
(43) Pub. Date: Dec. 19, 2024

(54) Continuous Flow Reactor and Process for Synthesis of Substituted Benzoic Acid

(71) Applicant: Council of Scientific and Industrial Research, New Delhi (IN)

(72) Inventors: Sunil Shankar JOSHI, Pune (IN); Nilesh Atmaram MALI, Pune (IN)

(73) Assignee: Council of Scientific and Industrial Research, New Delhi (IN)

(21) Appl. No.: 18/697,616

(22) PCT Filed: Oct. 3, 2022
(86) PCT No.: PCT/IN22/50881

§ 371 Date: Apr. 1, 2024

(30) Foreign Application Priority Data
Oct. 4, 2021 (IN) 202111045092

(51) Int. Cl. C07C 51/275; B01J 19/18

(52) U.S. Cl. CPC C07C 51/275; B01J 19/1856; B01J 2219/00033

(57) ABSTRACT

The present invention provides a non-hazardous, improved continuous oxidation process for the preparation of substituted benzoic acid of formula (I) by using continuous process reactor system with improved process controls. (I) wherein: R₁ is H, F, Cl, Br, I, NO₂, amine, or C₁-C₅ alkyl; R₂ is H, F, Cl, Br, I, NO₂, amine, or C₁-C₅ alkyl; and R₃ is H, F, Cl, Br, I, NO₂, amine, C₁-C₅ alkyl;

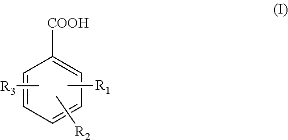

### FIELD OF THE INVENTION

The present invention relates to a continuous oxidation process for the synthesis of substituted benzoic acid using dilute nitric acid. Particularly, the present invention provides a non-hazardous, improved continuous oxidation process for the preparation of substituted benzoic acid by using continuous process reactor system with improved process controls.

More particularly, the present invention provides a continuous mode process reactor system for the preparation of substituted benzoic acid.

### BACKGROUND OF THE INVENTION

Conventionally, synthesis of substituted benzoic acid are carried out using oxidation of substituted toluene with oxidizing agents such as sodium dichromate, potassium permanganate, manganese dioxide, and the like.

All the conventional methods include batch processes for this oxidation reaction leading the process hazardous on large scale.

References may be made to PCT application WO2007/122638A2 entitled “An improved process for preparation of lamotrigine intermediates” provides a batch process for the synthesis of 2,3-dichlorobenzoic acid by oxidation of 2,3-dichlorotoluene using dilute nitric acid.

References may be made to PCT application WO2002/30862A1 entitled “Oxidation of alkyl-aromatic compounds” reports a process for the oxidation of an alkyl-aromatic compound, wherein the aromatic compound is admixed with an oxidizing agent or sulfur compound in the presence of an ionic liquid.

These processes have several limitations such as the high cost of the oxidizing agents, huge effluent generation and the difficulties in purifying the final product due to formation of unknown side products. Also, these processes are carried out in batch reactors in which it is difficult to manage heat of reaction and there is a risk of run-away situation leading to hazards due to large chemical inventory.

Therefore, there is a need in the art to provide non-hazardous, continuous process reactor system for the production of benzoic acid derivatives through oxidation process of toluene derivatives.

### Objectives of the Invention

Main objective of the present invention is to provide a continuous oxidation process for the preparation of substituted benzoic acid by using continuous process reactor system with improved process controls with zero effluent generation.

Another objective of the present invention is to provide a continuous process reactor system for the preparation of substituted benzoic acid.

### SUMMARY OF THE INVENTION

Accordingly, present invention provides a non-hazardous and continuous oxidation process for the preparation of substituted benzoic acid compound of formula (I)

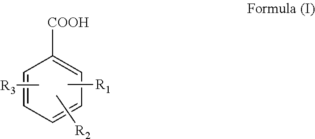

wherein:
R₁ is H, F, Cl, Br, I, NO₂, amine, or C₁-C₅ alkyl;
R₂ is H, F, Cl, Br, I, NO₂, amine, or C₁-C₅ alkyl; and
R₃ is H, F, Cl, Br, I, NO₂, amine, C₁-C₅ alkyl;
using continuous process reactor system comprising a step of:
   i. oxidizing compound of formula (A) with oxidizing agent in a continuous reactor at a temperature in the range of 150-180° C. and pressure in the range of 15-25 bar to obtain the compound of formula (I) with 95-100% selectivity.

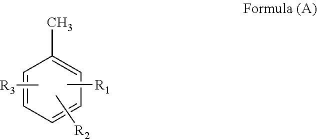

wherein R₁-R₃ are as defined earlier.

In an embodiment of the present invention, the oxidizing agent is selected from the group consisting of dilute nitric acid, dilute nitric acid with phase transfer catalyst or hydrogen peroxide solution.

In another embodiment of the present invention, said process further comprises mixing oxidizing agent with a surfactant.

In yet another embodiment of the present invention, the surfactant is anionic surfactants or cationic surfactants selected from the group consisting of dodecyl trimethylammonium chloride, dimethylhexadecylamine oxide, dimethyloctylamine oxide, dimethyldodecyl amine oxide, dihydroxyethyldodecylamine oxide or dimethyltetradecylamidopropyl amine oxide.

In yet another embodiment of the present invention, the temperature is in the range of 150-180° C.

The process as claimed in claim 1, wherein the continuous process reactor is selected from the group consisting of coiled tubular reactor, shell and tube reactor configuration or double pipe reactor configuration.

In yet another embodiment, present invention provides a continuous process of synthesis of substituted benzoic acid compound of formula (I) using continuous process reactor comprising the step of:
(a) pumping nitric acid and de-mineralized water from dosing tanks to the acid dilution vessel wherein flow of pumps are set such to form dilute nitric acid of concentration in the range of 20-25 weight %;
(b) pumping dilute nitric acid as formed in step (i) and compound of formula (A) from the respective tanks in the weight ratio ranging between 3:1 to 5:1 into the preheating tanks;
(c) preheating the reactants in the preheaters followed by mixing in the static mixers to obtain mixed reactant stream;
(d) flowing the mixed reactant stream as obtained in step (c) to reactors, connected in parallel in a down-flow manner, maintaining the temperature of the reactors at a temperature level of 150-180° C. by removing heat of the reaction with thermic fluid;
(e) receiving the outlet of reactors containing product, impurities along with unreacted reactants into the reaction crude receiver and venting the NOx in the reaction crude through a pressure regulating valve;
(f) filtering the reaction crude slurry comprising solid product to recover the solid products;
(g) phase separating the filtrate in a phase separator to provide organic stream separated from the aqueous stream; and
(h) recycling the organic phase, aqueous phase and nitric acid regenerated from effluent NO_{X}, back to the reactor system.

In yet another embodiment, present invention provides a continuous process reactor system for synthesis of substituted benzoic acid compound of formula (I) comprising:
i. reactants feed vessels (HV-01 and HV-02) to pump the nitric acid and DM water respectively to an acid dilution vessel (MV-01) using conc. acid feed pump (P-01) and DM water feed pump (P-02);
ii. reactants feed vessel (HV-03) to pump the compound of formula (A) using reactants feed pumps (P-03A-D, P-04A-D);
iii. reactants preheaters (HE-01-HE-08) to preheat the reactants from the acid dilution vessel (MV-01) and reactants feed vessel (HV-03);
iv. static mixers (MX-01-MX-04) to mix the preheated reactants;
v. jacketed tubular reactors (RE-01-RE-04) for reacting the reactants in a temperature ranging between 150 to 180° C.;
vi. reactor crude collection vessels (MV-02A-B) to collect the crude product with impurities;
vii. continuous filtration system (F-01) for filtering the product;
viii. phase separator (PS-01) to provide organic stream separated from the aqueous stream; and
ix. aqueous effluent receiver vessel (HV-05), organic layer transfer pump (P-05), aqueous recycle pump (P-06), organic receiver vessel (HV-04) and organic recycle pump (P-07) for recycling the organic phase, aqueous phase and nitric acid regenerated from effluent NO_{X}, back to reactor system.

In yet another embodiment, present invention provides a the jacketed tubular reactors (RE-01-RE-04) maintains pressure using a back-pressure regulator valve (V-01) in the vapor line from a reactor effluent receiver (MV-02A-B).

In yet another embodiment, present invention provides a said reactor further comprises a gas scrubbing system to capture a gas stream exiting the reactor system which comprises:
i. scrubbing solvent feed vessel (MV-03) to provide solvent feed using scrubbing solvent feed pump (P-08) to a scrubbing column (C-01);
ii. an air feeding unit (AU-01) to provide air to the scrubbing column (C-01) and scrubbed gas exits from top of the scrubbing column (C-01);
iii. a bottom receiver vessel (HV-06) to receive the stream through a scrubber bottom transfer pump (P-09) and the stream from the receiver vessel (HV-06) is exited through a scrubber bottom receiver (P-10).

### BRIEF DESCRIPTION OF THE DRAWINGS

FIG. 1 depicts the flow diagram of a continuous process reactor system used for said oxidation process to produce substituted benzoic acid.

FIG. 2 represents HPLC data for 2,3-DCBA [2,3-dichlorobenzoic acid].

FIG. 3 depicts the flow diagram for continuous process for synthesis of 2,3-DCBA from 2,3-DCT.

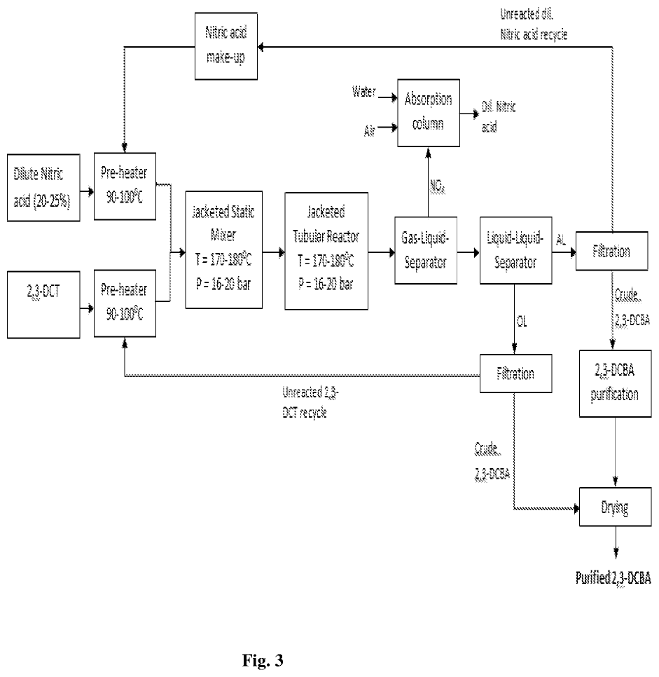

FIG. 4 depicts the process step for the preparation of substituted benzoic acid compound of formula (I).

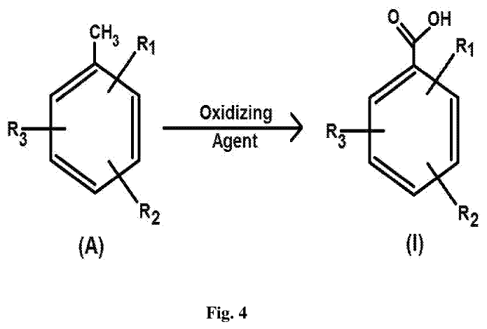

### DETAILED DESCRIPTION OF THE INVENTION

The present invention provides a non-hazardous, zero-effluent, improved continuous oxidation process for the preparation of substituted benzoic acid by using continuous process reactor system with improved process controls.

The present invention provides a non-hazardous, continuous oxidation process for the preparation of substituted benzoic acid by using continuous process reactor system, wherein said continuous process reactor system comprises of: reactants feed vessels (HV-01-HV-03), conc. acid feed pump (P-01), demineralized [DM] water feed pump (P-02), acid dilution vessel (MV-01), reactants feed pumps (P-03A-D, P-04A-D), reactants preheaters (HE-01-HE-08), static mixers (MX-01-MX-04), jacketed tubular reactors (RE-01-RE-04), reactor crude collection vessels (MV-02A-B), continuous filtration system (F-01), phase separator (PS-01), aqueous effluent receiver vessel (HV-05), organic layer transfer pump (P-05), aqueous recycle pump (P-06), organic receiver vessel (HV-04) and organic recycle pump (P-07). The pressure in the reactor system is mainlined by a back-pressure regulator valve (V-01) in the vapor line from the reactor effluent receiver (MV-02A-B). The gas stream exiting the reactor system may be required to capture in a suitable solvent system.

A gas scrubbing system is comprises of scrubbing solvent feed vessel (MV-03), scrubbing solvent feed pump (P-08), air feeding unit (AU-01), scrubbing column (C-01), scrubber bottom transfer pump (P-09) and scrubber bottom receiver (P-10). The gases from reaction crude receivers (MV-02A/B) are sent to scrubber column C-01 where is it is contacted with scrubbing solvent in countercurrent manner. The scrubbed gases exit from top of the column and scrubber bottom stream is transferred to bottom receiver vessel HV-06.

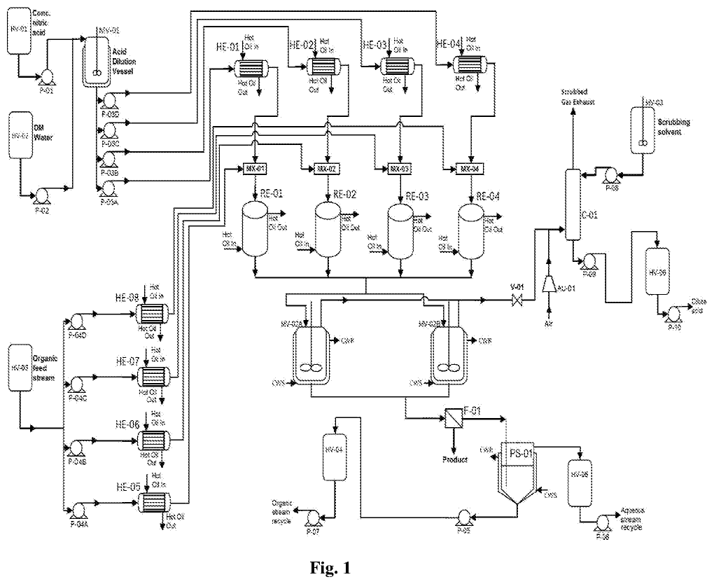

Here it is important to note that although in the embodiment shown in FIG. 1 comprised of four reactors with corresponding four preheaters, static mixers and feed pumps, as per the scale of operation, the number of reactors can decrease or increase.

The present invention provides a non-hazardous, zero-effluent continuous oxidation process for the preparation of substituted benzoic acid compound of formula (I) by using continuous process reactor system

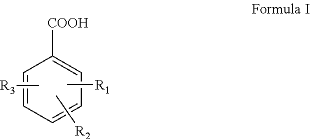

wherein
R₁ is H, F, Cl, Br, I, NO₂, amine, or C₁-C₅ alkyl;
R₂ is H, F, Cl, Br, I, NO₂, amine, or C₁-C₅ alkyl; and
R₃ is H, F, Cl, Br, I, NO₂, amine, or C₁-C₅ alkyl;
and the said continuous process comprises of:
   i. oxidizing compound of formula (A) with oxidizing agent in a continuous reactor at a temperature in the range of 150-180° C. and pressure in the range of 15-25 bar to afford the compound of formula (I) with 95-100% selectivity.

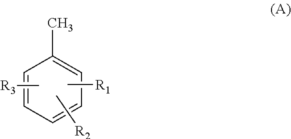

wherein R₁-R₃ is as defined earlier.

The present invention relates to a continuous process of synthesis of substituted benzoic acid compound of formula (I) using tubular reactor, shell and tube reactor or double pipe reactor,

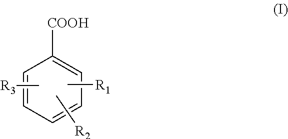

wherein R₁-R₃ is as defined earlier.
and the continuous process comprising the step of:
   a) pumping nitric acid and de-mineralized water from dosing tanks to the acid dilution vessel wherein the flows of pumps are set such that dilute nitric acid of concentration in the range of 20-25 weight % is formed;
   b) pumping dilute nitric acid (20-25 weight %) and compound of formula (A) from the respective tanks in the ratio of 3:1 to 5:1 (by weight) into the preheaters;
   c) preheating the reactants in the preheaters before mixing in the static mixers;
   d) flowing the mixed reactant stream to reactors, connected in parallel in a down-flow manner, the temperature of the reactors being maintained at a temperature level of 150-180° C. by removing heat of the reaction with thermic fluid;
   e) receiving the outlet of reactors containing product, impurities along with unreacted reactants into the reaction crude receiver and venting the NOx in the reaction crude through a pressure regulating valve;
   f) filtering the reaction crude slurry comprising solid product to recover the solid products;
   g) phase separating the filtrate in a phase separator to provide organic stream separated from the aqueous stream; and
   h) recycling the organic phase, aqueous phase and nitric acid regenerated from effluent NO_{X}, back to reactor system.
   i) In yet another aspect, the present invention relates to a continuous process reactor system for synthesis of substituted benzoic acid compound of formula (I) comprising of:
   j) reactants feed vessels (HV-01 and HV-02) to pump the nitric acid and DM water to an acid dilution vessel (MV-01) using acid feed pump (P-01) and DM water feed pump (P-02);
   k) reactant feed vessel (HV-03) to pump the compound of formula (A) using reactants feed pumps (P-03A-D, P-04A-D);
   l) reactants preheaters (HE-01-HE-08) to preheat the reactants from the acid dilution vessel (MV-01) and reactant feed vessel (HV-03),
   m) static mixers (MX-01-MX-04) to mix the preheated reactants;
   n) jacketed tubular reactors (RE-01-RE-04) for reacting the reactants in a temperature range of 150° C. to 180° C.;
   o) reactor crude collection vessels (MV-02A-B) to collect the crude product with impurities;
   p) continuous filtration system (F-01) for filtering the product;
   q) phase separator (PS-01) to provide organic stream separated from the aqueous stream; and
   r) aqueous effluent receiver vessel (HV-05), organic layer transfer pump (P-05), aqueous recycle pump (P-06), organic receiver vessel (HV-04) and organic recycle pump (P-07) for recycling the organic phase, aqueous phase and nitric acid regenerated from effluent NO_{X}, back to reactor system.

The continuous process reactor system further comprises a gas scrubbing system to capture a gas stream exiting the reactor system, wherein the gas scrubbing system comprises:
(a) scrubbing solvent feed vessel (MV-03) to provide solvent feed using scrubbing solvent feed pump (P-08) to a scrubbing column (C-01);
(b) an air feeding unit (AU-01) to provide air to the scrubbing column (C-01) and scrubbed gas exits from top of the scrubbing column (C-01); and
(c) a bottom receiver vessel (HV-06) to receive the stream through a scrubber bottom transfer pump (P-09) and the stream from the receiver vessel (HV-06) is exited through a scrubber bottom receiver (P-10).

Present invention provides the use of surfactant to mix aqueous nitric acid and compound of formula (A), which reduces the colored impurities of the product.

Present invention provides recycle of organic and aqueous streams from the reactor outlet which lead to zero discharge process.

Suitable oxidizing agent is selected from the group comprising of dilute nitric acid, dilute nitric acid with phase transfer catalyst, hydrogen peroxide solution preferably dilute nitric acid.

Suitable continuous reactors used for the reaction are selected from coiled tubular reactor, Shell and tube reactor configuration, double pipe reactor configuration preferably coiled tubular reactor.

The continuous process provides 95-100% selectivity of the benzoic acid compound of formula (I).

The temperature in the reactor is in the range of 170° C. to 180° C., preferably 175° C. to 180° C.

The present invention provides the use of surfactant to mix aqueous nitric acid and compound of formula (A), which reduces the colored impurities of the product.

The surfactant is anionic surfactants, cationic surfactants or combination thereof.

Surfactants are selected from the group comprising of anionic surfactants, cationic surfactants such as dodecyl trimethylammonium chloride, aliphatic amine oxide surfactants such as dimethylhexadecylamine oxide, dimethyloctylamine oxide, dimethyldodecyl-amine oxide, dihydroxyethyldodecylamine oxide, dimethyltetradecylamidopropyl amine oxide.

The present invention provides provision for recycle of organic, aqueous and gaseous streams from reactor outlet which leads to zero-effluent process.

FIG. 1 depicts the flow diagram of a continuous process reactor system used for said oxidation process to produce benzoic acid derivatives. As FIG. 1 shows process for the preparation of 2, 3-dichlorobenzoic acid as a representative example with four reactors in the reactor system. The number of reactors in the reactor system can decrease or increase as per the desired production capacity. Conc. Nitric acid and DM water are pumped from the dosing tanks HV-01 and HV-02, respectively to the acid dilution vessel MV-01. Flows of pump P-01 and P-02 are set such that dilute nitric acid of concentration in the range of 20-25 weight % is formed in the MV-01 tank. Dilute nitric acid (20-25 weight %) and 2,3-DCT [2,3-dichlorotoluene] are pumped from the tanks MV-01 and HV-03, respectively in the ratio of 3:1 to 5:1 (by weight) using the pumps P-03A-D and P-04A-D, respectively. The reactants are preheated in the preheaters to around 130-140° C. before getting mixed in the static mixers MX-01-04. 2,3-DCT feed stream is preheated in preheaters HE-05-08, whereas dilute nitric acid stream is preheated in preheaters HE-01-04. The mixed reactant stream flows to the four tubular reactors RE-01, RE-02, RE-03 & RE-04, connected in parallel in a down-flow manner. The reactor jackets are provided with thermic fluid heating loop with a provision for both heating and cooling. Thermic fluid is used as heating and cooling utility. Reaction is expected to kick-off with initial heating in the preheaters. Once reaction picks-up well, with the exotherm of the reaction, the temperature of the reactor can be maintained at the desired temperature level of 150-180° C. by removing heat of reaction with thermic fluid.

Outlet of all four reactors containing product, impurities along with unreacted reactants is sent to the reaction crude receiver MV-02. NOx in the reaction crude is vented through a pressure regulating valve (V-01) and are scrubbed in the scrubber C-01 with DM water as solvent. Air from air handling unit AU-01 is mixed in the NOx stream before it enters the scrubber C-01. The reaction crude slurry comprising solid product is then filtered in a filter F-01 to recover the solid products. The remaining filtrate is then sent to a phase separator PS-101 for separating organic stream from the aqueous stream. Organic stream being heavier will be collected in the bottom section of the phase separator and is transferred to the hold tank HV-04 using the transfer pump P-07. In continuous operation, organic phase is recycled back to reactor system through pump P-07. The aqueous stream from the phase separator overflows to the aqueous hold tank HV-05. The aqueous stream is recycled back to the reactor system using pump P-06 through acid dilution vessel MV-01, wherein desired feed concentration of dilute nitric acid is maintained after recycle stream is added. To maintain the reaction system pressure to the desired values of 15-25 bar(a), a Pressure Regulating Valve (V-01) is installed in the vapor line from the reaction crude receiver, MV-02A-B.

### NOx Scrubbing

NOx is generated during the reaction and are separated from the reaction mixture in the reaction crude receiver MV-02. NOx gases flow to the scrubber C-01 where it is scrubbed with DM water as solvent. Small quantity of air is added in the NOx gaseous stream through air handling unit AU-01, before it enters the scrubber to convert NO to NO₂. Scrubbed exhaust gases are released to the atmosphere and dilute nitric acid obtained in the scrubber bottom is sent to aqueous stream receiver HV-06.

The continuous process reactor system for synthesis of substituted benzoic acid compound of formula (I) comprising:
(a) reactants feed vessels (HV-01 and HV-02) to pump the nitric acid and DM water to an acid dilution vessel (MV-01) using conc. acid feed pump (P-01) and DM water feed pump (P-02);
(b) reactants feed vessel (HV-03) to pump the compound of formula (A) using reactants feed pumps (P-03A-D, P-04A-D);
(c) reactants preheaters (HE-01-HE-08) to preheat the reactants from the acid dilution vessel (MV-01) and reactants feed vessel (HV-03);
(d) static mixers (MX-01-MX-04) to mix the preheated reactants;
(e) jacketed tubular reactors (RE-01-RE-04) for reacting the reactants in a temperature range of 150 to 180 C;
(f) reactor crude collection vessels (MV-02A-B) to collect the crude product with impurities;
(g) continuous filtration system (F-01) for filtering the product;
(h) phase separator (PS-01) to provide organic stream separated from the aqueous stream; and
(i) aqueous effluent receiver vessel (HV-05), organic layer transfer pump (P-05), aqueous recycle pump (P-06), organic receiver vessel (HV-04) and organic recycle pump (P-07) for recycling the organic phase, aqueous phase and nitric acid regenerated from effluent NO_{X}, back to reactor system.

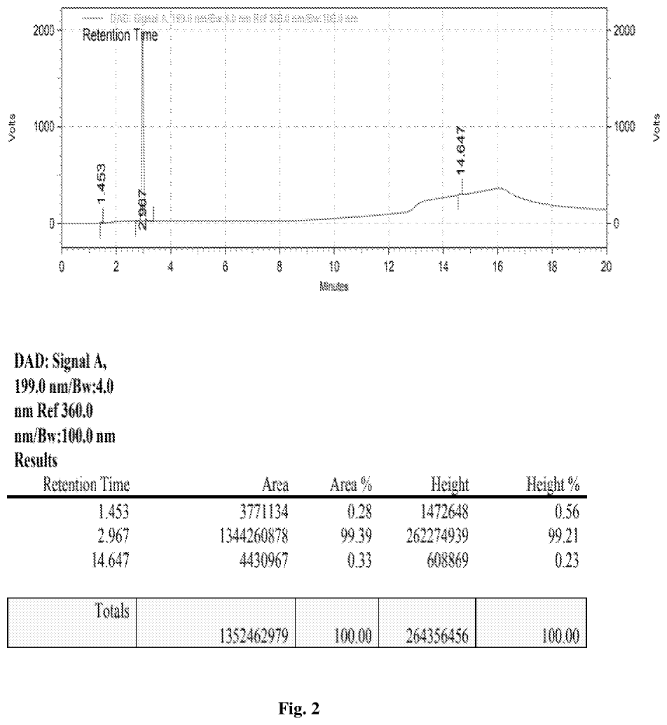

FIG. 2 depicts HPLC result for 2,3-DCBA, showing 99.39% purity with 2,3-DCBA's retention time of 2.967 minutes.

Several experiments are conducted at different reactor conditions to obtain 2,3-DCBA as a representative product starting with 2,3-DCT. Results are summarized below in Table-1.

TABLE-1

| | Reactor | | Feed flow | | | | | |
|---|---|---|---|---|---|---|---|---|
| | conditions | | | HNO₃ | | | 2,3- | |
| | | Process | 2,3- | (22 | | Residence | DCBA | |
| Sr. | P | T | DCT | mass %) | Conversion | time | Selectivity | |
| No | (bar) | (° C.) | (ml/min) | (ml/min) | (%) | (min) | (%) | Remarks |
| 1 | 20 | 175 | 5 | 30 | 74.47 | 29 | 95.07 | Surfactant |
| | | | | | | | | conc. = 1.25% |
| 2 | 21 | 174 | 2.5 | 15 | 89.69 | 57 | 95.82 | Surfactant |
| | | | | | | | | conc. = 1.25% |
| 3 | 18 | 175 | 2.5 | 16.5 | 72.57 | 31 | 94.54 | Surfactant |
| | | | | | | | | conc. = 1.25% |
| 4 | 18 | 174 | 2.5 | 15 | 76.16 | 26 | 96.78 | Surfactant |
| | | | | | | | | conc. = 0.625% |
| 5 | 18 | 176 | 2.5 | 15 | 100 | 29 | 94.56 | Without |
| | | | | | | | | Surfactant |
| 7 | 20 | 178 | 2.5 | 15 | 100 | 39 | 95.34 | Recycle; |
| | | | | | | | | without |
| | | | | | | | | surfactant |
| 6 | 16 | 180 | 2.5 | 6.25 | 41.4 | 114 | 100 | Surfactant |
| | | | | | | | | conc. = 1.5% |

### EXAMPLES

Following examples are given by way of illustration and therefore should not be construed to limit the scope of the invention.

### Example 1: General Process for the Preparation of Compound of Formula (I)

Compound of formula (A), was oxidized with dilute nitric acid as oxidizing agent (22 weight %) in a continuous tubular reactor at 175-180° C. and 15-25 bar(a) pressure. Both reactants were fed continuously using pump to the tubular coil reactor which was already heated to temperature up to 175-180° C. by circulating hot oil through the reactor jacket. The oxidation products included majority of benzoic acid derivative of formula (I) with selectivity in the range of 95-100%. The reaction crude from the reactor system was collected in a tank and then filtered to remove the solid product. The aqueous and organic phases were separated in a phase separator. The organic phase mainly comprised of unreacted compound of formula (A) which was stored in a hold tank for recycling while aqueous stream containing unreacted nitric acid was stored in another hold tank.

### Example 2

Four liters of dilute nitric acid of 22% concentration (by mass) is prepared and stored in a dosing vessel. 1 liter of 2,3-dichlorotoluene of purity around 98% (by mass) is fed to another dosing vessel. First complete reactor system was flushed with nitrogen gas. Then feed of dilute nitric acid was started at 15 ml/min using a feed pump and simultaneously heating of the preheaters and the coiled reactor initiated by starting the hot oil circulation. Once preheaters and reactor are preheated, feed of 2,3-DCT at 2.5 ml/min was started using another feed pump. Reaction pressure increases with the temperature. The process temperature and pressure were controlled at 180° C. and 20 bar, respectively. The excess pressure was released through a Pressure Regulating Valve in the vapor line of the reaction mass collection vessel at the outlet of the reactor. Once sufficient reaction mass was collected in the collection vessel, reaction was stopped by stopping the feeds and cooling was started and pressure was released to the scrubber. The reaction mass was then sent to phase separator in which organic and aqueous layer was separated and filtered to recover solid product, crude 2,3-DCBA. Crude 2,3-DCBA was then given several washings with water and then dried under vacuum at around 80° C. to get final dry product. Both, organic and aqueous layer obtained after filtration can be recycled to with make-up of 2,3-DCT and dilute nitric acid to match the desired flow rates to the reactor.

### Example 3

Similar process as that mentioned in example 2 was run with 2,4-dichlorotoluene as starting material. Dilute nitric acid of strength 22 mass % was fed at 15 ml/min rate along with 2,4-dichlorotoluene feed at the rate of 2.5 ml/min. The process temperature was controlled at a temperature of 175° C. and a pressure of 18 bar. Crude 2,4-DCBA was formed which was collected and purified in a similar way as that mentioned in example 2 for 2,3-DCBA. Yield more than 90% was obtained for 2,4-DCBA.

### Advantages of the Invention

The process of the present invention provides reduced inventory to improve safety in operations.
The process of the present invention provides significantly less unknowns in the products.
The process of the present invention is significantly intensified process as compared to batch process for production capacity.
The process of the present invention is zero-effluent/zero-discharge process as both organic, aqueous and gaseous streams from reactor outlet are recycled.

1-10. (canceled)

11. A non-hazardous and continuous oxidation process for preparing a substituted benzoic acid compound of formula (I): 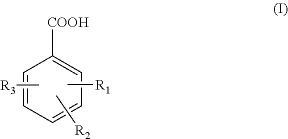
where:
R₁ is H, F, Cl, Br, I, NO₂, amine, or C₁-C₅ alkyl;
R₂ is H, F, Cl, Br, I, NO₂, amine, or C₁-C₅ alkyl; and
R₃ is H, F, Cl, Br, I, NO₂, amine, C₁-C₅ alkyl;
the process comprising:
oxidizing, by a continuous process, a compound of formula (A): 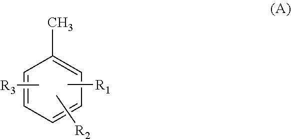
where R₁, R₂, and R₃ are as defined in formula (I), with an oxidizing agent in a continuous process reactor at a temperature from 150° C. to 180° C. and a pressure from 15 bar to 25 bar to obtain the compound of formula (I) with 95% to 100% selectivity.

12. The process according to claim 11, wherein the oxidizing agent is selected from the group consisting of dilute nitric acid, dilute nitric acid and a phase transfer catalyst, and a hydrogen peroxide solution.

13. The process according to claim 11, further comprising mixing the oxidizing agent with a surfactant.

14. The process according to claim 13, wherein the surfactant is an anionic surfactant or a cationic surfactant and is selected from the group consisting of dodecyl trimethylammonium chloride, dimethylhexadecylamine oxide, dimethyloctylamine oxide, dimethyldodecyl amine oxide, dihydroxyethyldodecylamine oxide, and dimethyltetradecylamidopropyl amine oxide.

15. The process according to claim 11, wherein the continuous process reactor is selected from the group consisting of a coiled tubular reactor, a shell-and-tube reactor configuration, or double pipe reactor configuration.

16. The process according to claim 11, wherein the continuous process comprises:
(a) pumping nitric acid and de-mineralized water from dosing tanks to an acid dilution vessel by pumps having a flow set so as to form dilute nitric acid having a concentration from 20% to 25% by weight;
(b) pumping the dilute nitric acid of (a) and the compound of formula (A) from respective tanks into preheating tanks at a weight ratio of dilute nitric acid to compound of formula (A) from 3:1 to 5:1;
(c) preheating the reactants in the preheating tanks, then mixing the reactants in static mixers, to obtain a mixed reactant stream;
(d) flowing the mixed reactant stream of (c) to reactors, the reactors being connected in parallel in a down-flow manner, while maintaining the reactors at a temperature from 150° C. to 180° C. by removing heat of the reaction with thermic fluid;
(e) receiving an outlet from the reactors, the outlet containing product, impurities, and unreacted reactants, into a reaction crude receiver, and venting NOx in the reaction crude receiver through a pressure regulating valve;
(f) filtering reaction crude slurry comprising solid product to recover solid products;
(g) phase separating filtrate in a phase separator to provide an organic stream separated from an aqueous stream; and
(h) recycling back to the continuous process reactor: organic phase in the organic stream, aqueous phase in the aqueous stream, and nitric acid regenerated from effluent NO_{X}.

17. A continuous process reactor system for synthesizing a substituted benzoic acid compound of formula (I): 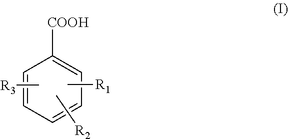
where:
R₁ is H, F, Cl, Br, I, NO₂, amine, or C₁-C₅ alkyl;
R₂ is H, F, Cl, Br, I, NO₂, amine, or C₁-C₅ alkyl; and
R₃ is H, F, Cl, Br, I, NO₂, amine, C₁-C₅ alkyl;
the continuous process reactor system comprising:
a first reactants feed vessels to pump nitric acid and de-mineralized water to a first acid dilution vessel using a concentrated acid feed pump and a de-mineralized water feed pump;
a second reactants feed vessel to pump, using reactants feed pumps, a compound of formula (A): 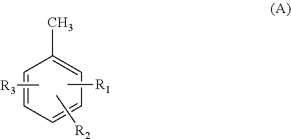
where R₁, R₂, and R₃ are as defined in formula (I);
a second acid dilution vessel to pump dilute acid using the feed pumps;
reactants preheaters to preheat reactants from the first acid dilution vessel and the second reactants feed vessel;
static mixers to mix the preheated reactants;
jacketed tubular reactors for reacting the reactants at a temperature from 150° C. to 180° C.;
reactor crude collection vessels to collect crude product with impurities;
a continuous filtration system for filtering the product;
a phase separator to provide organic stream separated from the aqueous stream; and
an aqueous effluent receiver vessel, an organic layer transfer pump, an aqueous recycle pump, an organic receiver vessel, and an organic recycle pump for recycling organic phase, aqueous phase, and nitric acid regenerated from effluent NO_{X}, back to the continuous process reactor system.

18. The continuous process reactor system according to claim 18, wherein the jacketed tubular reactors maintain pressure using a back-pressure regulator valve in a vapor line from a reactor effluent receiver.

19. The continuous process reactor system according to claim 18, further comprising a gas scrubbing system to capture a gas stream exiting the reactor system, the gas scrubbing system comprising:
(i) a scrubbing solvent feed vessel to provide solvent feed using scrubbing solvent feed pump to a scrubbing column;
(ii) an air feeding unit to provide air to the scrubbing column and scrubbed gas exits from top of the scrubbing column;
(iii) a bottom receiver vessel to receive the stream through a scrubber bottom transfer pump and the stream from the receiver vessel is exited through a scrubber bottom receiver.
